(19) United States
(12) Patent Application Publication
(10) Pub. No.: US 2011/0183993 A1
(43) Pub. Date: Jul. 28, 2011

(54) CYCLOALKANE DERIVATIVE

(75) Inventors: Ryu Nagata, Osaka (JP); Mayumi Oda, Fukuoka (JP); Norihiko Tanno, Osaka (JP); Masayuki Muto, Osaka (JP); Ikutaro Saji, Osaka (JP)

(73) Assignee: Dainippon Sumitomo Pharma Co., Ltd., Osaka (JP)

(21) Appl. No.: 13/015,745

(22) Filed: Jan. 28, 2011

Related U.S. Application Data
(60) Provisional application No. 61/298,987, filed on Jan. 28, 2010.

(51) Int. Cl. A61K 31/496; C07D 417/14; A61P 25/18

(52) U.S. Cl. 514/254.04; 544/368

(57) ABSTRACT

The present invention relates to a novel cycloalkane derivative which has an excellent psychotropic action.

### TECHNICAL FIELD

The present invention relates to a novel cycloalkane derivative and an acid addition salt thereof which are useful as a psychotropic drug. In more detail, the present compound is useful as a medicament for treating, for example, schizophrenia, senile psychiatric disorder, bipolar disorder, neurosis, and associated symptoms of senile dementia.

### BACKGROUND ART

Patent Reference 1 discloses some cycloalkane derivatives which have psychotropic action. Although the disclosed derivatives have the same chemical structures as the present invention, Patent Reference 1 fails to disclose a compound having the steric configurations of formulae [1]-[3] or formulae [X1]-[X4] shown herein.

Furthermore, psychotropic drugs which have been currently used can be accompanied with some adverse events such as side effects in CNS, including extrapyramidal adverse effects (e.g. catalepsy), oversedation, and cognitive dysfunction. Consequently, such adverse events have been a serious problem clinically.

### PRIOR ART

### Patent Reference

[Patent Reference 1] JP-5 (1993)-17440 A (U.S. Pat. No. 5,532,372 A, EP 464846 A1)

### DISCLOSURE OF INVENTION

### Problem to be Solved by the Invention

The purpose of the present invention is to provide a good psychotropic drug. In particular, the purpose is to provide a psychotropic drug which exhibits an excellent effect for improving a broad spectrum of schizophrenia such as positive symptom, negative symptom, and cognitive dysfunction.

### Means to Solve the Problem

The present inventors have extensively studied to reach the above object and then have found that a cycloalkane derivative having a novel steric configuration exhibits the desired various pharmacological actions. Based upon the new findings, the present invention has been completed.

The present invention relates to the following inventions:

### Term 1

A compound of formula [1]:

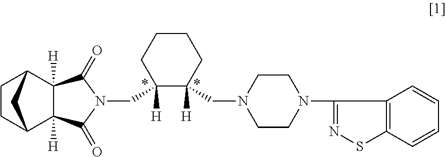

or formula [2]:

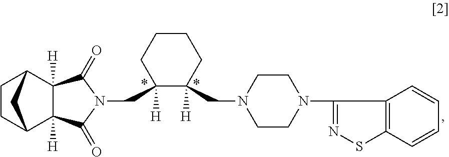

or an acid addition salt thereof.

### Term 2

The compound of Term 1 wherein the structure of the compound is formula [1]:

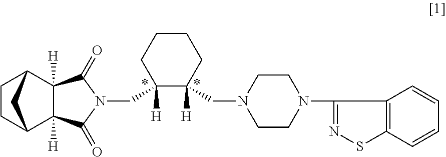

or an acid addition salt thereof.

### Term 3

The compound of Term 1 wherein the structure of the compound is formula [2]:

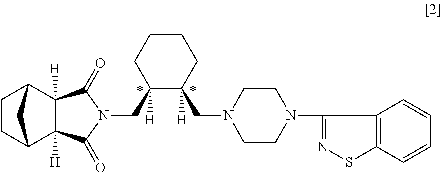

or an acid addition salt thereof.

### Term 4

A compound of formula [X1]:

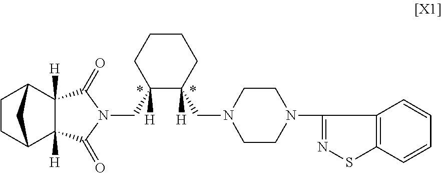

or formula [X2]:

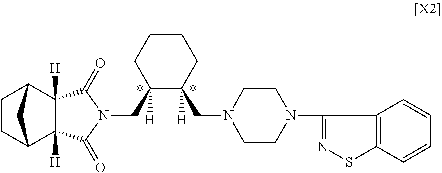

or an acid addition salt thereof.

### Term 5

The compound of Term 4 wherein the structure of the compound is formula [X1]:

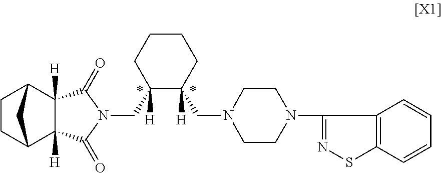

or an acid addition salt thereof.

### Term 6

The compound of Term 4 wherein the structure of the compound is formula [X2]:

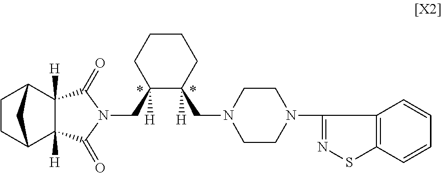

or an acid addition salt thereof.

### Term 7

A compound of formula [X3]:

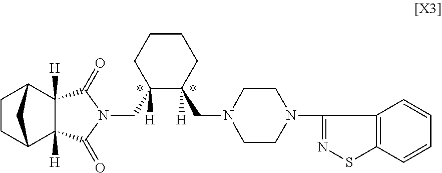

or formula [X4]:

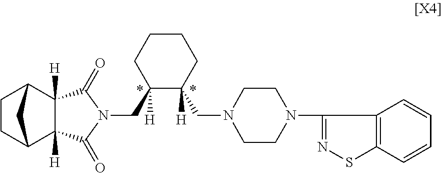

or an acid addition salt thereof.

### Term 8

The compound of Term 7 wherein the structure of the compound is formula [X3]:

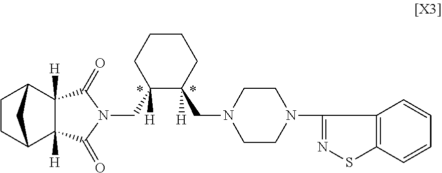

or an acid addition salt thereof.

### Term 9

The compound of Term 7 wherein the structure of the compound is formula [X4]:

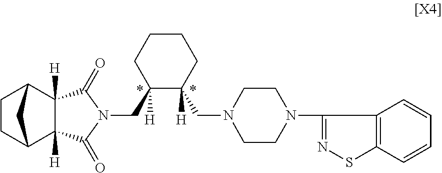

or an acid addition salt thereof.

### Term 10

An antipsychotic agent comprising the compound of any one of Terms 1-9 or an acid addition salt thereof.

### Term 11

A method for treating psychosis comprising administering an effective amount of the compound of any one of Terms 1-9 or an acid addition salt thereof to a mammal in need thereof.

### Term 12

Use of the compound of any one of Terms 1-9 or an acid addition salt thereof in the preparation of an antipsychotic agent.

### Term 13

An agent for treating schizophrenia comprising the compound of any one of Terms 1-9 or an acid addition salt thereof.

### Term 14

A compound of formula [3]:

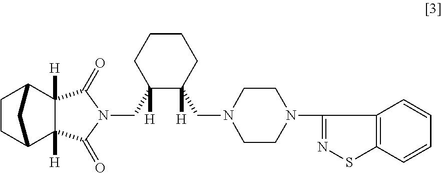

or an acid addition salt thereof.

### BEST MODE FOR CARRYING OUT THE INVENTION

The present compounds can exist as a hydrate and/or solvate and hence also include such hydrate and/or solvate thereof.

The present compounds include a mixture of the above-defined compounds [1] and [2], [X1] and [X2], and [X3] and [X4], respectively, with a constant ratio.

The acid additive salt used herein includes an addition salt with a pharmaceutically acceptable inorganic acid or organic acid. The salt with an inorganic acid includes, for example, hydrochloride, hydrobromide, hydroiodide, sulfate, nitrate, phosphate, etc. and preferably hydrochloride. The salt with an organic acid includes, for example, acetate, oxalate, citrate, malate, tartrate, maleate, fumarate, etc.

The present compounds [1] and [2] can be obtained by, for example, resolving a racemic form of compound [4] (disclosed as compound number 126 in Patent Reference 1) using HPLC on an optically active column. The structure of compound [4] is as follows:

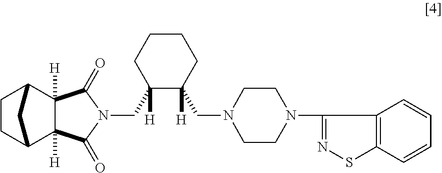

The optically active column should not be limited to a particular optically active column as long as compounds [1] and [2] can be isolated. However, the optically active columns are preferably normal-phase systems, and specifically, for example, DICEL CHIRALCEL OJ-H, DICEL CHIRALCEL OD-H, YMC CHIRAL NEA(R), etc.

The present compound [3] can be synthesized by, for example, coupling compound [5] with compound [6] (disclosed as compound numbers 255 and 206 in Patent Reference 1, respectively) using the synthetic conditions described in Patent Reference 1 which discloses some syntheses for a compound similar to compound [3].

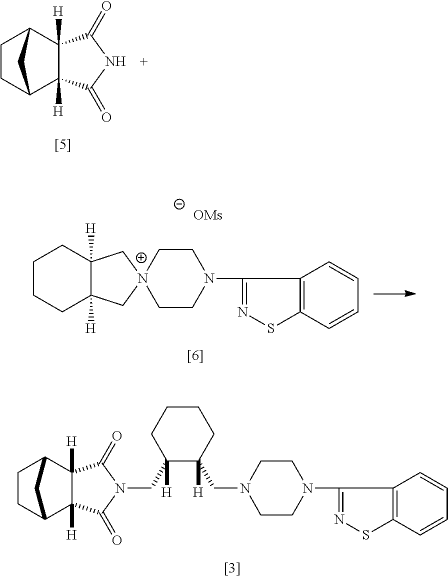

The racemic compound [3] can be resolved using HPLC on an optically active column to give the present compounds [X1] and [X2].

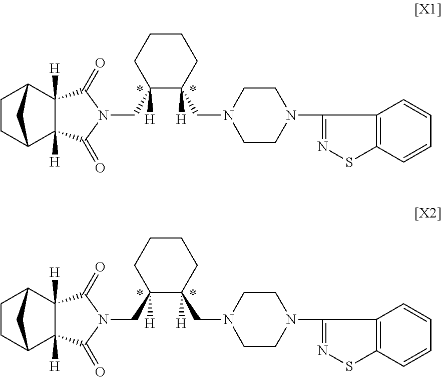

The optically active column used herein, should not be limited to a particular optically active column as long as compounds [X1] and [X2] can be isolated, includes the above-exemplified optically active columns for optically-resolving compound [4].

The racemic compound [10] can be synthesized by, for example, coupling compound [5] with compound [9] (disclosed as compound numbers 255 and 201 in Patent Reference 1, respectively) using the synthetic conditions described in Patent Reference 1 which discloses some syntheses for a compound similar to compound [10].

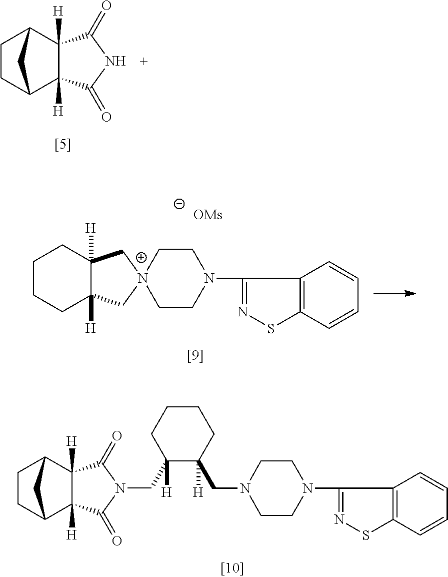

The racemic compound [10] can be resolved using HPLC on an optically active column to give the present compounds [X3] and [X4].

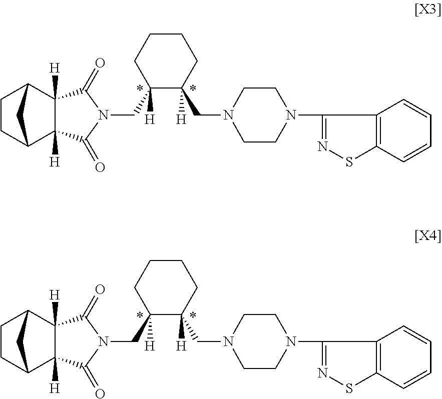

The optically active column used herein, should not be limited to a particular optically active column as long as compounds [X3] and [X4] can be isolated, includes the above-exemplified optically active columns for optically-resolving compound [4].

In addition, the present compounds [X3] and [X4] can also be obtained by using an optically-active compound as a starting material. For example, the desired compounds can be synthesized by preparing bicycloheptane-dicarboximide moiety, an optically active cyclohexane-dicarboxylic acid derivative moiety and a 3-(1-piperazinyl-1,2-benzisothiazole moiety, and then coupling them together in the same manner as shown in Patent Reference 1.

Furthermore, the racemic compounds [4], [3] and [10] can be optically resolved on a conventional method, i.e., by forming a salt thereof with an optically active acid such as L-tartaric acid and D-tartaric acid, separating it through fractional crystallization, and making it desalted.

The present compounds can be administered orally or parenterally in the medical use. Namely, the compounds can be orally administered as a generally-used dosage form such as powder, granule, tablet, capsules, syrup, and suspension, or parenterally administered as an injection form such as solution, emulsion, and suspension thereof. And it can be rectally administered as a suppository. Furthermore, it can be intravesically administered as a solution. The above-mentioned dosage form can be prepared by formulating the present compounds with conventional additives such as carrier, excipient, binder, stabilizer, and diluent. In the case of injections, for example, acceptable buffer, solubilizer, and isotonic agent can be also used. In the case of the above-mentioned oral formulation or suppository, the present compounds may be contained preferably in 0.1-70% (w/w) per the composition. The dosage and the frequency of administration depend on various conditions such as target disease, symptom, age and body weight of a subject, type of formulation, and manner of administration. In general, the present compounds can be administered in a dosage of 0.1-2000 mg, preferably 1-200 mg per a day for an adult, and once to several times (e.g. twice to 4 times) a day.

The present compounds are useful for treating psychosis, in more detail as follows.

The present compounds exhibit high affinity for one or multiple subtypes of various receptors, for example, dopaminergic receptors such as dopamine D₁ receptor, dopamine D₂ receptor, dopamine D₃ receptor and dopamine D₄ receptor; serotonergic receptors such as serotonin 5-HT_{1A} and serotonin 5-HT₂; and noradrenergic receptors such as α₁ noradrenergic receptor and α₂ noradrenergic receptor.

It has been well known that D₂ receptor antagonistic action is strongly correlated with psychotic effect (see: e.g. Seeman, Pharmacol. Rev., 32, 229 (1981)). And also, it has been reported that 5-HT₂ receptor antagonistic action is useful for antipsychotic effect (see: e.g. Janssen et al., J. Pharm. Exper. Ther., 244, 685 (1988)). In particular, D₂ receptor antagonistic action can control positive symptoms of schizophrenia (e.g. hallucination, delusion), while 5-HT₂ receptor antagonistic action can contribute to improve negative symptoms of schizophrenia (e.g. indifference, social withdrawal). In addition, it has been suggested that 5-HT₂ receptor antagonistic action can decrease some extrapyramidal adverse events which often arises in a maintenance therapy of schizophrenia using a D₂ receptor antagonist.

In addition, it has been suggested that D₄ antagonistic action which is one of other dopaminergic receptor subtypes does not cause extrapyramidal adverse events which often arise in a maintenance therapy of schizophrenia (see, e.g. Seeman et al., Nature, 350, 610 (1991); Seeman et al., Nature, 358, 149 (1992)).

Further, it has been reported that antagonistic action of 5-HT_{1A} receptor which is another subtype of serotonergic receptors is correlated with antianxiety (see: e.g. Titeler, Biochem. Pharmacol., 36, 3265 (1987)).

Accordingly, the present compounds have psychotropic actions such as antipsychotic action, antianxiety, and antidepressive action, which are useful, for example, as a medicament for treating schizophrenia, senile psychiatric disorder, bipolar disorder, neurosis, and associated symptoms of senile dementia, etc.

### EXAMPLES

Hereinafter, the present invention is further illustrated by Reference examples, Examples, and Tests, but should not be construed to be limited thereto. In addition, the triangular solid lines and hash lines used herein denote an absolute configuration and the bold solid lines and hash lines used herein denote a relative configuration.

### Example 1

Using compound [6] and compound [7] (disclosed as compound numbers 206 and 251 in Patent Reference 1, respectively), compound [8] was prepared as a racemate (about 20 mg) by following the process shown in Example 1-(a) of Patent Reference 1.

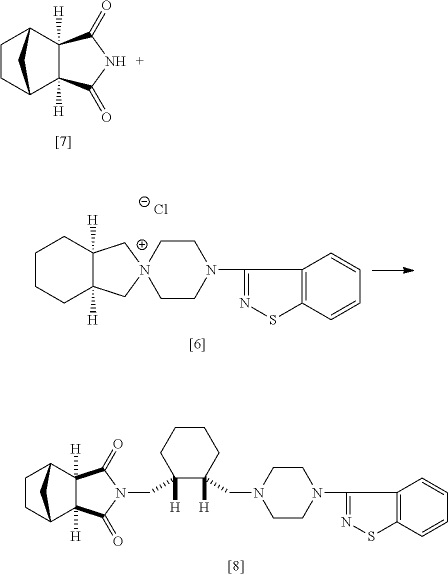

¹H-NMR (Hydrochloride of compound [8], DMSO-d₆, 300 MHz) δ: 9.91 (bs, 1H), 8.15 (d, 1H, J=8.3 Hz), 8.11 (d, 1H, J=8.3 Hz), 7.61 (t, 1H, J=7.6 Hz), 7.48 (t, 1H, J=7.6 Hz), 4.07 (bt, 2H, J=10.8 Hz), 3.57-3.70 (m, 2H), 3.14-3.57 (m, 8H), 2.75 (s, 2H), 2.15 (bs, 1H), 1.91 (bs, 1H), 1.47-1.70 (m, 6H), 1.20-1.70 (m, 8H), 1.16 (d, 1H, J=10.6 Hz), 0.95 (d, 1H, J=10.6 Hz).

m.p. (Hydrochloride of compound [8]): 235-237° C.

The resulting racemic compound [8] (20 mg) was resolved on HPLC under the following conditions to give optical isomer 1-A (9 mg) and optical isomer 1-B (9 mg). In addition, although the absolute configurations of the two optical isomers were not identified, one was compound [1] and the other was compound [2].

### HPLC Resolution Conditions

Column: DICEL CHIRALCEL OJ-H (2 cm I.D.×25 cm)

Mobile phase: n-hexane/ethanol/diethylamine=50/50/0.1 (v/v/v)

Flow rate: 10 ml/min

Column temperature: 40° C.

Detection wavelength: 254 nm

### Retention Time

Optical isomer 1-A: 12.6 min

Optical isomer 1-B: 19.2 min

The resulting optical isomers 1-A and 1-B were analyzed on HPLC under the following conditions shown in “HPLC Analytic Conditions” and “Retention Time” to confirm the results of “Optical Purity” which is also shown below.

### HPLC Analytic Conditions

Column: DICEL CHIRALCEL OJ-H (0.46 cm I.D.×25 cm)

Mobile phase: n-hexane/ethanol/diethylamine=50/50/0.1 (v/v/v)

Flow rate: 1.0 ml/min

Column temperature: 40° C.

Detection wavelength: 254 nm

### Retention Time

Optical isomer 1-A: 7.67 min

Optical isomer 1-B: 11.71 min

### Optical Purity

Optical isomer 1-A: not less than 98% ee

Optical isomer 1-B: not less than 98% ee

### Example 2

Using compound [5] (2.26 g) and compound [6] (disclosed as compound numbers 255 and 206 in Patent Reference 1, respectively), compound [3] is prepared as a racemate (5.185 g) by following the process shown in Example 1-(a) of Patent Reference 1.

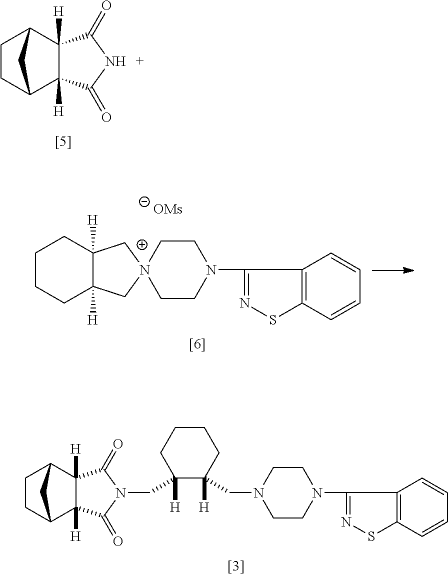

¹H-NMR (CDCl₃, 300 MHz) δ: 7.91 (d, 1H, J=8.1 Hz), 7.80 (d, 1H, J=8.1 Hz), 7.46 (t, 1H, J=8.0 Hz), 7.35 (t, 1H, J=8.0 Hz), 3.45-3.62 (m, 5H), 3.40 (dd, 1H, J=4.5, 13.2 Hz), 3.10 (bd, 2H, J=17 Hz), 2.70 (bd, 2H, J=17 Hz), 2.50-2.70 (m, 4H), 2.30-2.50 (m, 2H), 2.07 (bs, 1H), 1.92 (bs, 1H), 1.45-1.80 (m, 6H), 1.15-1.45 (m, 8H).

The resulting racemic compound [3] was resolved on HPLC under the following conditions to identify optical isomer 3-A and optical isomer 3-B. In addition, although the absolute configurations of the two optical isomers were not identified, one was compound [X1] and the other was compound [X2].

### HPLC Analytic Conditions

Column: YMC CHIRAL NEA(R) (0.46 mm I.D.×25 cm, used as two of which are serially connected)

Mobile phase: 0.1 mol/L sodium perchlorate solution (pH 3.0)/acetonitrile=50:50 (v/v)

Flow rate: 1.2 ml/min

Column temperature: 35° C.

Detection wavelength: 210 nm

### Retention Time

Optical isomer 3-A: 40.8 min

Optical isomer 3-B: 41.6 min

### Example 3

Using compound [5] (1.1 g) and compound [9] (2.56 g) (disclosed as compound numbers 255 and 201 in Patent Reference 1, respectively), compound [10] was prepared as a racemate (2.657 g) by following the process shown in Example 1-(a) of Patent Reference 1.

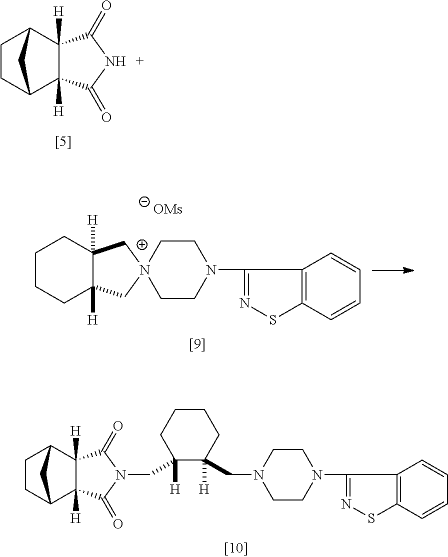

¹H-NMR (CDCl₃, 300 MHz) δ: 7.91 (d, 1H, J=8.1 Hz), 7.80 (d, 1H, J=8.1 Hz), 7.46 (t, 1H, J=8.0 Hz), 7.35 (t, 1H, J=8.0 Hz), 3.94 (dd, 1H, J=3.3, 13.2 Hz), 3.53 (bs, 4H), 3.32 (dd, 1H, J=9.5, 13.2 Hz), 3.07 (bs, 2H), 2.76 (bs, 2H), 2.55-2.70 (m, 6H), 2.24 (dd, 1H, J=6.6, 12.2 Hz), 1.89 (bd, 1H, J=12.2 Hz), 1.50-1.75 (m, 6H), 1.0-1.50 (m, 8H).

The resulting racemic compound [10] was resolved on HPLC under the following conditions to give optical isomer 10-A (2.2 mg) and optical isomer 10-B (1.9 mg). In addition, although the absolute configurations of the two optical isomers were not identified, one was compound [X3] and the other was compound [X4].

### HPLC Resolution Conditions

Column: DICEL CHIRALCEL OD-H (0.46 cm I.D.×25 cm)

Mobile phase: acetonitrile/diethylamine=100:0.1 (v/v)

Flow rate: 1.0 ml/min

Column temperature: 40° C.

Detection wavelength: 318 nm

### Retention Time

Optical isomer 10-A: 5.131 min

Optical isomer 10-B: 5.934 min

The resulting optical isomers 10-A and 10-B were analyzed on HPLC under the following conditions shown in “HPLC Analytic Conditions” and “Retention Time” to confirm the results of “Optical Purity” which is also shown below.

### HPLC Analytic Conditions

Column: DICEL CHIRALCEL OD-H (0.46 cm I.D.×25 cm)

Mobile phase: acetonitrile/diethylamine=100:0.1 (v/v)

Flow rate: 1.0 ml/min

Column temperature: 40° C.

Detection wavelength: 318 nm

### Retention Time

Optical isomer 10-A: 5.134 min

Optical isomer 10-B: 5.935 min

### Optical Purity

Optical isomer 10-A: not less than 98% ee

Optical isomer 10-B: not less than 98% ee

### Test 1. D₂ Receptor Binding Assay

According to the known method (e.g. Japan. J. Pharmacol., 53, 321-329 (1990)), the above-captioned experiment was carried out using [³H] spiperone, i.e., the binding amount of [³H] spiperone to the cell membrane preparation expressing human D₂ receptor was measured, and then the binding inhibitory rate by the test compound (10 nM) was measured/calculated. The table below shows the results in IC₅₀ values, which were obtained by calculating %-inhibitory concentration according to Hill analysis (see, Hill A. V., J. Physiol., 40, 190-200 (1910)).

| | IC₅₀ (nM) |
|---|---|
| Optical isomer 1-A | 17.9 |
| Optical isomer 1-B | 3.24 |
| Compound [10] | 3.26 |

### Test 2. 5-HT₇ Receptor Binding Assay

To a buffer solution containing 50 mM Tris-HCl (pH=7.6), 4 mM CaCl₂ and 0.5 mM EDTA were added 50 μl of [³H] 5-CT (final concentration: 0.5 nM), 1 μl of a solution of the test compound, and 150 μl of h5-HT₇/CHO membrane preparation (the total amount of the reaction medium: 201 μl). Using the reaction medium, the receptor binding activity of each test compound was measured. The reaction medium was incubated at room temperature for 40 minutes, and then quickly filtrated under reduced pressure through a glass-fiber filter. The glass-fiber filter was washed twice with 200 μl of 50 mM Tris-HCl (pH=7.6), and then put into a counting vial containing 4 ml of ACS-II (Amersham), and the receptor binding radioactivity of the residue on the filter was measured with a liquid scintillation counter. And the radioactivity was measured in the presence of 10 μM SB-269970 (5-HT₇ receptor ligand). Based on the above results, the binding inhibitory rate of each test compound (100 nM) was calculated. The table below shows the results in IC₅₀ values, which were obtained by calculating 50%-inhibitory concentration according to Hill analysis (see, Hill A. V., J. Physiol., 40, 190-200 (1910)).

| | IC₅₀ (nM) |
|---|---|
| Optical isomer 1-A | 154 |
| Optical isomer 1-B | 18.3 |
| Compound [10] | 2.66 |

1. A compound of formula [1]: 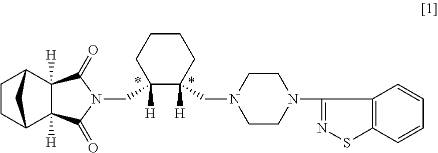
or formula [2]: 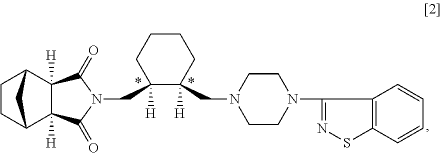
or an acid addition salt thereof.

2. The compound of claim 1 wherein the structure of the compound is formula [1]: 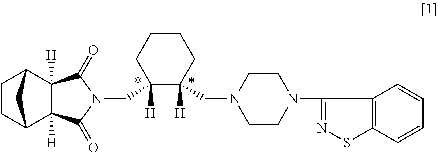
or an acid addition salt thereof.

3. The compound of claim 1 wherein the structure of the compound is formula [2]: 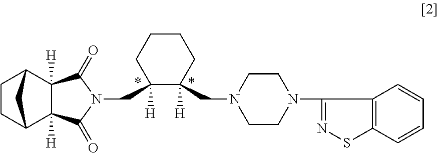
or an acid addition salt thereof.

4. A compound of formula [X1]: 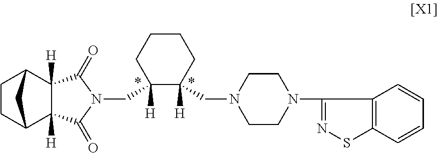
or formula [X2]: 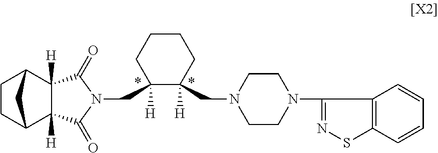
or an acid addition salt thereof.

5. The compound of claim 4 wherein the structure of the compound is formula [X1]: 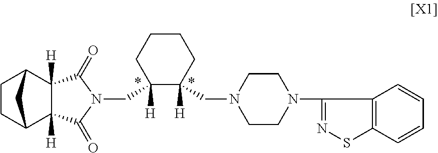
or an acid addition salt thereof.

6. The compound of claim 4 wherein the structure of the compound is formula [X2]: 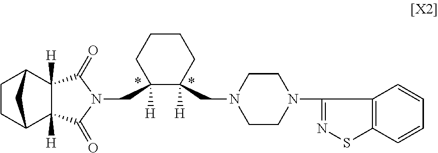
or an acid addition salt thereof.

7. A compound of formula [X3]: 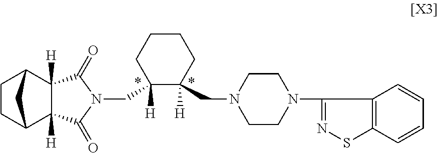
or formula [X4]: 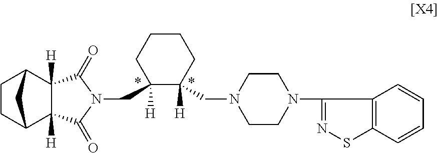
or an acid addition salt thereof.

8. The compound of claim 7 wherein the structure of the compound is formula [X3]: 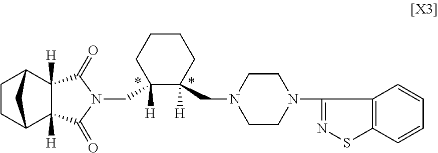
or an acid addition salt thereof.

9. The compound of claim 7 wherein the structure of the compound is formula [X4]: 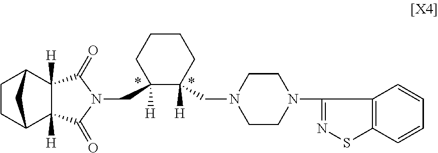
or an acid addition salt thereof.

10. An antipsychotic agent comprising the compound of any one of claims 1, 4 or 7 or an acid addition salt thereof.

11. A method for treating psychosis comprising administering an effective amount of the compound of any one of claims 1, 4 or 7 or an acid addition salt thereof to a mammal in need thereof.

12. (canceled)

13. An agent for treating schizophrenia comprising the compound of any one of claims 1, 4 or 7 or an acid addition salt thereof.
